(19) United States
(12) Patent Application Publication
(10) Pub. No.: US 2013/0031809 A1
(43) Pub. Date: Feb. 7, 2013

(54) SHOE HAVING IMPROVED PODIATRIC SUPPORT

(71) Applicants: Patricia CALFEE, San Francisco, CA (US); Ian PICACHE, San Francisco, CA (US)

(72) Inventors: Patricia CALFEE, San Francisco, CA (US); Ian PICACHE, San Francisco, CA (US)

(73) Assignee: Roses & Rye LLC, San Francisco, CA (US)

(21) Appl. No.: 13/204,409

(22) Filed: Aug. 5, 2011

(51) Int. Cl. A61F 5/14

(52) U.S. Cl. 36/144

(57) ABSTRACT

Disclosed herein are variations of shoes having structural features that offer improved podiatric support to address any number of lower extremity pathologies. Such shoes are especially useful in allowing individuals adapt to physical changes, accompanying ailments, as well as lifestyle changes that can arise such as those during and after pregnancy, during motherhood or other situations that induce temporary or permanent physical changes.

### CROSS-REFERENCES MADE TO RELATED APPLICATIONS

Not applicable.

### STATEMENT AS TO RIGHTS TO INVENTIONS MADE UNDER FEDERALLY SPONSORED RESEARCH AND DEVELOPMENT

Not applicable.

### FIELD OF THE INVENTION

The present disclosure includes shoes having structural features that offer improved podiatric support to address any number of lower extremity pathologies. Such shoes are especially useful in allowing individuals adapt to physical changes, accompanying ailments, as well as lifestyle changes that can arise such as those during and after pregnancy, during motherhood or other situations that induce temporary or permanent physical changes.

### BACKGROUND OF THE INVENTION

The devices of the present disclosure can help to prevent or provide relief from foot, leg, knee, hip and lower back pain. While such relief may be sought among women during and after pregnancy as well as throughout motherhood, the devices and methods described herein can be used by any number of individuals seeking relief from foot, leg, knee, hip, lower back, and other types of pain. Accordingly, while the present disclosure discusses changes occurring during and after pregnancy, the inventive concepts can be applied to other situations in addition to pregnancy and post pregnancy users. A normal human gait begins with the outside part of the heel making contact with the ground. The foot then rolls inward, making complete contact with the ground. The inward rolling of the foot optimally distributes the forces of impact and is called pronation. Pronation is critical to shock absorption. Pushing off the forefoot region ends the gait cycle. Due to an increase in body weight and other physiological changes that occur during pregnancy as well as carrying young children during motherhood, women can experience a shifting of their center of gravity, altering their posture and normal gait.

Overpronation is a common foot problem associated with pregnancy and carrying young children during motherhood. Overpronation can result from excessive inward rolling of the foot. This may occur while a woman is stationary, or when walking after impact with the ground, such that the foot continues to roll inward during subsequent push-off. Because of the excessive inward rolling, the foot and ankle provide inadequate support and stabilization for the rest of the body and shock is not absorbed as efficiently. Thus, overpronation may be the source of many lower extremity pathologies, e.g., muscle tiredness and inflammation, foot and knee joint pain, back pain, plantar fasciitis, tendonitis and ligament strain.

Plantar fasciitis refers to an inflammation of the thick tissue on the bottom of the foot. Although many times a result of overpronation, women before and after pregnancy as well as carrying young children during motherhood may experience plantar fasciitis also from the sudden increase and redistribution of weight. Overpronation flattens the foot, putting additional strain on the plantar fascia which runs below the foot, from the metatarsalphalangeal joint to the calcaneus, or heel bone. Currently, the only treatment for plantar fasciitis is pain control medication, non-use, and/or wearing insoles with additional arch support and shock absorption.

Another common effect of pregnancy is edema, or swelling, which may occur anywhere in the body, but typically presents itself in the legs and feet in. Edema is an abnormal accumulation of fluid beneath the skin or in one or more cavities of the body. This abnormal accumulation of fluid increases the size of the feet and legs, making it especially difficult for women during and after pregnancy to fit comfortably in a normal shoe. In addition, as these women spend more time in a vertical position throughout the day, the swelling may worsen, rendering a shoe that may have fit comfortably earlier in the day to be tight and uncomfortable as the swelling increases. Although open shoes such as sandals may present one solution to this problem, many open shoes do not provide enough support to other parts of the woman's foot. Adequate support is critical during and after pregnancy to insure a safe, stable interface between a woman's foot and the ground. Also, adequate support is necessary to avoid other foot problems, such as overpronation. Furthermore, open shoes are not always appropriate due to safety, weather, professional expectations, etc.

Many pregnant women and mothers also experience leg cramping, varicose veins, and/or pain in the knees, hips and back. These ailments may be a result of the previously described ailments, or may occur independently as a result of the numerous physiological changes that come with pregnancy and motherhood, especially while carrying young children.

The prior methods and apparatus to address the physical ailments that affect women during pregnancy are less than ideal in at least some circumstances. For example, one conventional maternity insole has a varus of 2 to 7 degrees at the heel. However, a varus (or angle) in the heel portion alone does not adequately prevent and/or address an inward rolling of a woman's midfoot and forefoot. Furthermore, an insole alone, whether removably or permanently attached to a shoe, does not address pain caused by edema of the foot.

Another conventional insole attempts to address the forward shifting in weight distribution a woman experiences during pregnancy. This insole tilts rearwardly, attempting to shift the woman's body weight backward. However, it has been found that an insole with an elevated forefoot relative to the heel results in increased back, hip, leg and foot pain, contrary to the desired results herein. Furthermore, an insole alone, whether removably or permanently attached to a shoe, does not address pain caused by edema of the foot.

Yet another conventional insole attempts to provide relief to plantar fasciitis by incorporating a cupped heel portion and a midfoot portion having a medial longitudinal arch support. However, medial support of the arch alone does not provide adequate support for a woman's foot, nor does it adequately address overpronation. Furthermore, an insole alone, whether removably or permanently attached to a shoe, does not address pain caused by edema of the foot.

In light of the above, there remains a need for an improved shoe and/or insole design.

### SUMMARY OF THE INVENTION

Embodiments of the present invention provide a shoe and/or insole having structural features that offer improved podiatric support to address any number of lower extremity pathologies. In many embodiments, the shoe comprises a forefoot section, a midfoot section and a heel section. The midfoot section may include a varus causing the midfoot section to slope from a medial side of the midfoot section to a lateral side of the midfoot section.

In many embodiments, the heel section May have a varus of 0 degrees.

In another aspect, the midfoot section has a varus between 1 and 5 degrees.

In many embodiments, the shoe further comprises a slip resistant outsole.

In many embodiments, the shoe further comprises a cushioned insole.

The shoe may also include elastic material to accommodate swelling of the foot. Additionally, the shoe may comprise a partial d'Orsay shape.

In some embodiments described herein, the midfoot section of the shoe may further comprise an arched portion. The arched portion may be configured to provide support for the medial longitudinal, lateral longitudinal and tranverse arches.

In many embodiments, the midfoot varus causes weight distribution from a medial side of the foot to a lateral side of the foot.

### BRIEF DESCRIPTION OF DRAWINGS

FIG. 1 illustrates a perspective side view of a foot.

FIG. 2A illustrates a posterior view of a person's lower legs and ankles in a normal standing position.

FIG. 2B illustrates a posterior view of a person's lower legs and ankles during overpronation.

FIG. 3A illustrates a top view of the shoe.

FIG. 3B illustrates a side view of the shoe, as viewed from the medial side.

FIG. 4A illustrates a perspective view of the insole of a right shoe, as viewed from the lateral side.

FIG. 4B illustrates a perspective view of a longitudinal cross-section of the insole of the shoe.

FIG. 4C illustrates a perspective view of the insole of a right shoe, as viewed from the medial side.

FIG. 5 illustrates a transverse cross-sectional view of the heel section.

FIG. 6 illustrates the varus of the midfoot section.

FIGS. 7A-7F illustrate a sequence of cross-sectional view of a right shoe as viewed anteriorly.

FIG. 8A illustrates a cross-sectional view of the sole of the shoe, as viewed from the medial side.

FIG. 8B illustrates a cross-sectional view of the sole of the shoe, as viewed from the lateral side.

### DETAILED DESCRIPTION OF THE INVENTION

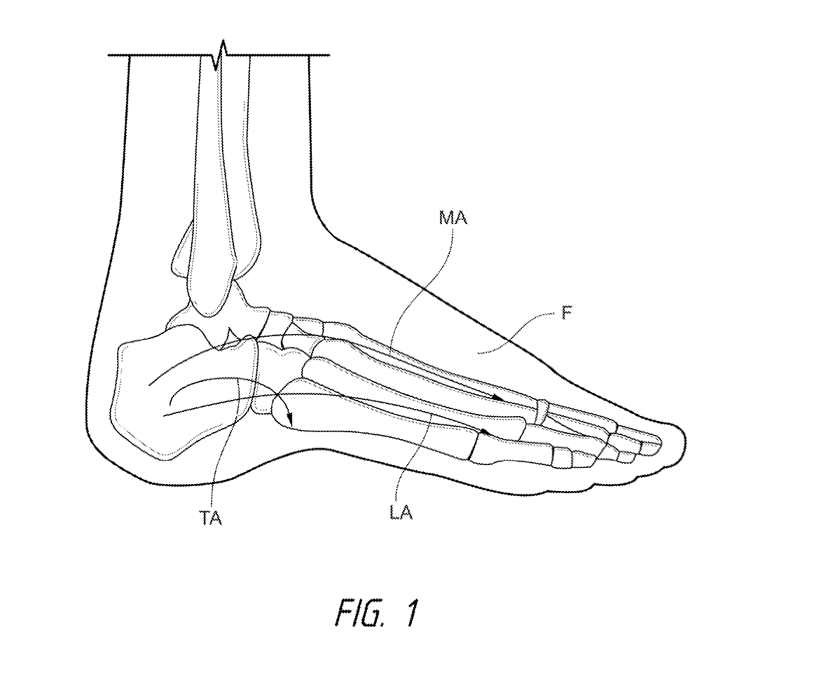

FIG. 1 illustrates a perspective side view of a foot F. Foot F has three arches, a medial longitudinal arch MA, a lateral longitudinal arch LA and a transverse arch TA. The medial longitudinal arch MA runs along the length of the arch on the inside of the foot. The lateral longitudinal arch LA runs along the length of the arch on the outside of the foot. The transverse arch TA runs widthwise across the arch medially to laterally. All three arches aid the foot in supporting and distributing a person's weight.

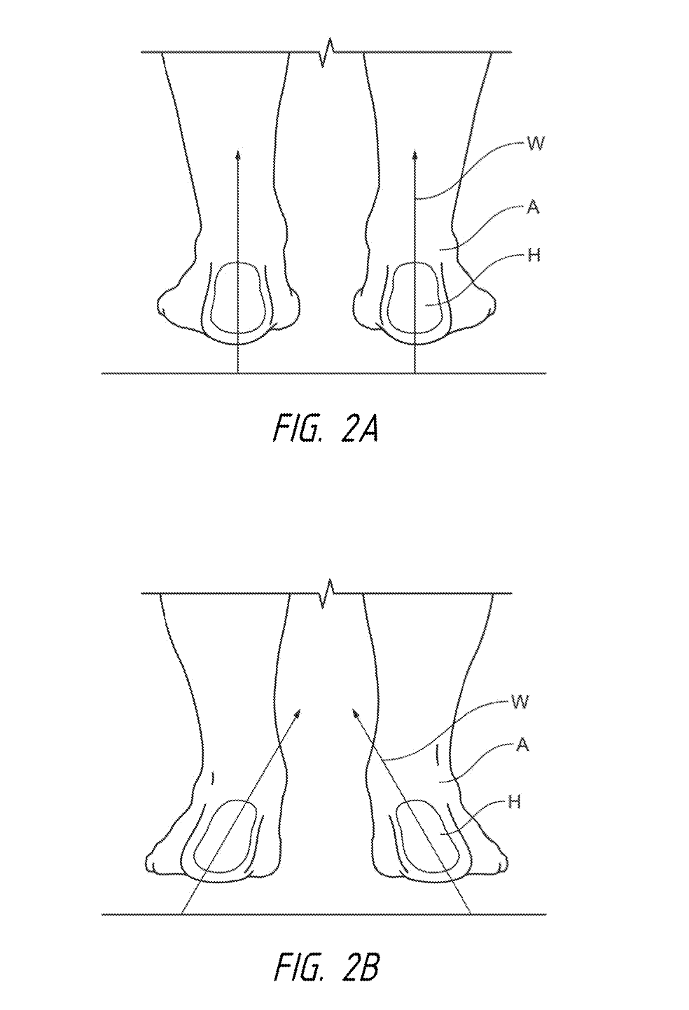

FIG. 2A illustrates posterior view of a person's lower legs and ankles A in a normal standing position, with a distribution of weight W centered on each heel H. FIG. 2B illustrates a posterior view of a person's lower legs and ankles A during excessive pronation, or overpronation, where the ankles and foot roll inward, resulting in an abnormal distribution of weight W, causing foot, leg, hip and back pain.

A person of ordinary skill in the art will recognize that the dimensions and physiology of the woman may vary, and although some women may comprise differing physiology, the teachings as described herein can be adapted for use by many women having various pregnancy and post-pregnancy conditions as well as varying foot dimensions. In addition, the designs described herein can be used for any individual suffering from similar conditions.

The devices described herein can be used to prevent or provide relief from foot, leg, knee, hip and lower back pain arising from conditions where the feet distribute weight in an unsatisfactory manner. For example, such conditions may arise during pregnancy.

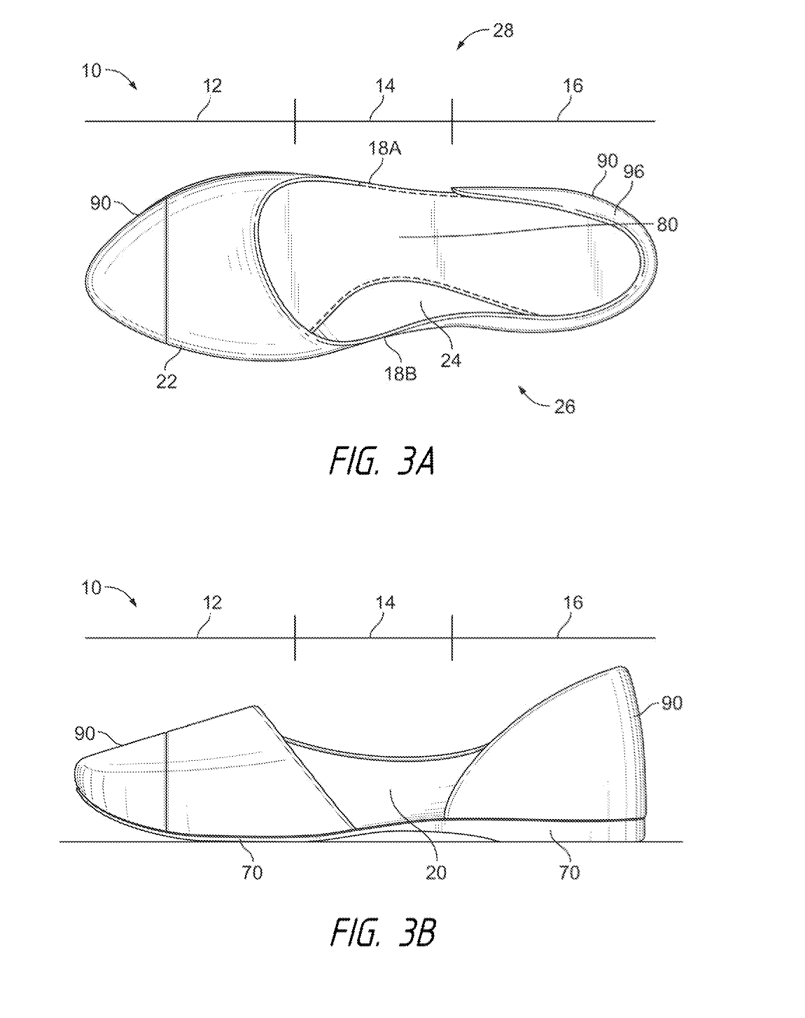

FIGS. 3A and 3B show top and side views of a shoe 10 comprising a forefoot section 12, a midfoot section 14, and a heel section 16. Each section 12, 14, 16 has an outsole 70, an insole 80, and a vamp 90. The outsole 70 and the insole 80 combine to make up the sole 60 of the shoe.

As shown in FIGS. 3A and 3B, sections 12, 14, 16 of the shoe can optionally include a vamp 90. The vamp 90 holds the shoe 10 onto the foot. In regard to the heel section 16, the vamp 90 may extend upwardly from the sole, creating a wall around the heel of the foot so as to protect the foot and preventing the foot from slipping out of the back of the shoe 10. In the midfoot section 14, the vamp 90 may extend upwardly, creating a wall along the Medial 26 and lateral 28 sides of the foot. In the forefoot section 12, the vamp 90 extends upwardly then inwardly over the forefoot portion of the foot, creating a protective housing over the forefoot. In addition to protecting the foot from the environment, the vamp 90 also provides structural support for the foot during the walking cycle. The vamp 90 can be made from any commercially available material known or used.

In one embodiment of the invention, the shoe 10 can optionally include a partial d'Orsay shape, such that there is a gap 18A in the vamp of the midfoot section 14, allowing an edemic foot to expand beyond the normal contours of the shoe. In another embodiment, an elastic wall 20 may span the gap in 18A. The elastic wall 20 provides additional support and protection to the foot, while still allowing the foot to comfortably expand if necessary. The elastic wall can be made of from any commercially available material.

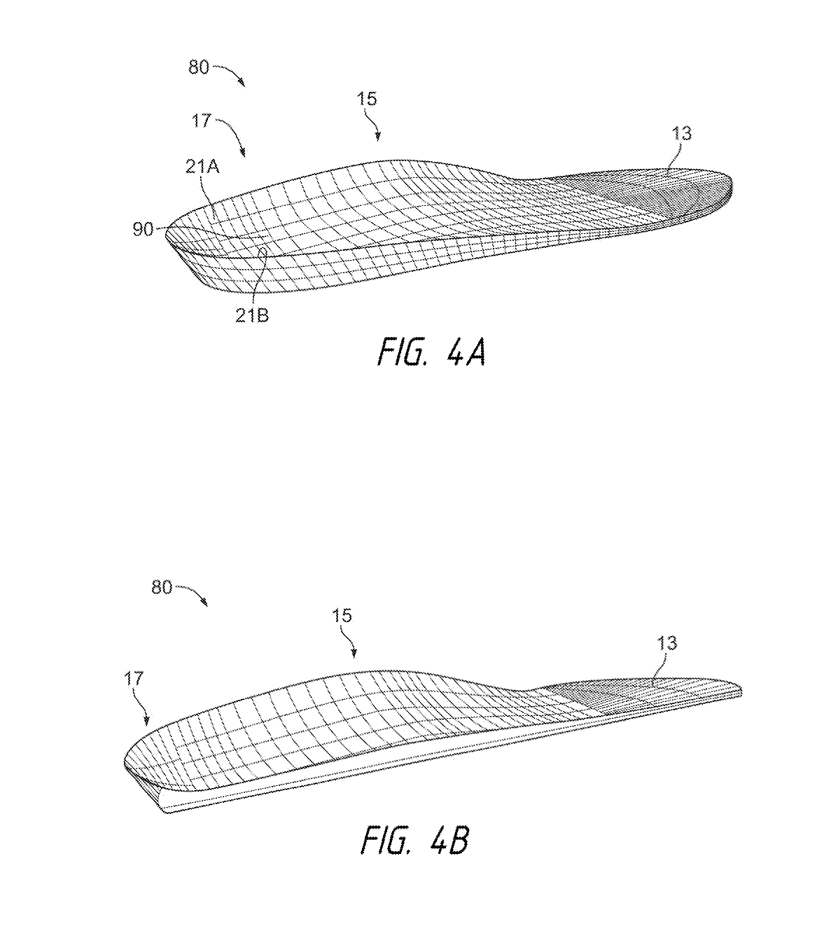

The forefoot section 12 comprises the anterior portion of the shoe. The forefoot section 12 may include a wide toe box 22 to further accommodate swelling of the foot. As shown in FIG. 4A, the forefoot section insole 13 is substantially flat.

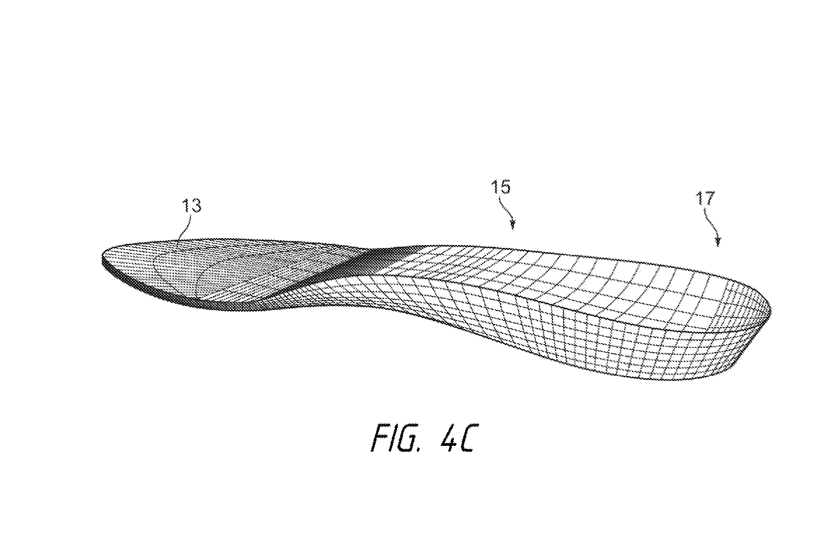

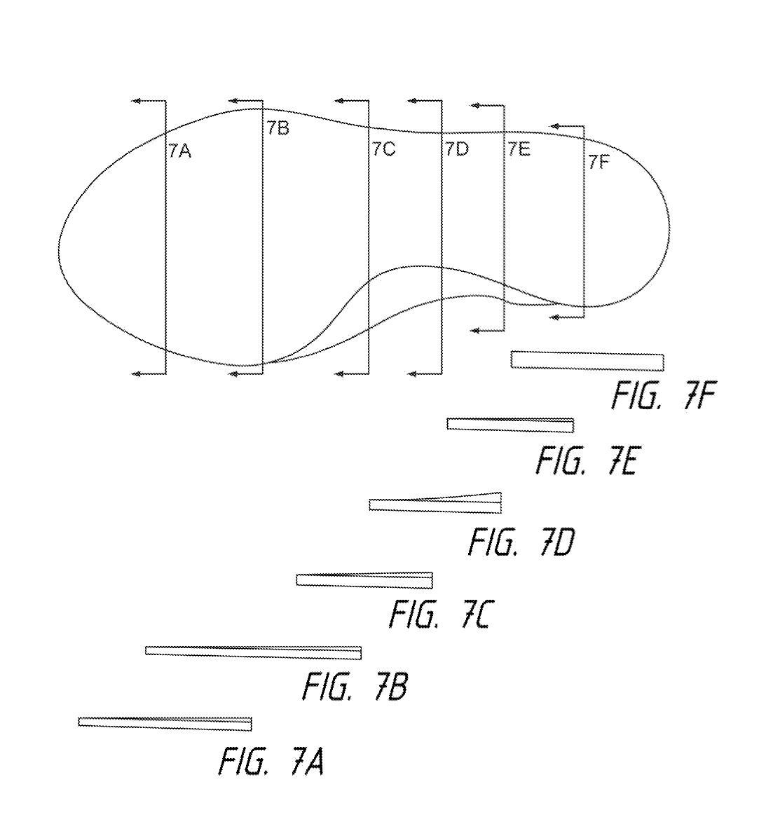

The heel section 16 makes up the posterior aspect of the shoe 10 and comprises a heel section insole 17 surrounded by an upwardly extending portion 96 of the vamp 90. The anterior portion of the heel section 16 extends into and is contiguous with the midfoot section 14. As shown in FIGS. 4A-4C, the heel section insole 17 may be slightly cupped, having a substantially flat center 19 surrounding by sloped side walls 21A and 21B. Alternatively, the heel section insole 17 may be flat along its entire width, as shown in the cross-section of FIG. 7F. FIG. 5 illustrates that although the heel section insole 17 may be cupped, the vertical axis of the heel section insole 17 and/or heel section 16 remains perpendicular to the ground plane G. This way, the heel section insole 17 and/or heel section 16 will not prejudice the heel of the foot towards with any degree varus, that is, deviation away from the midline of the body.

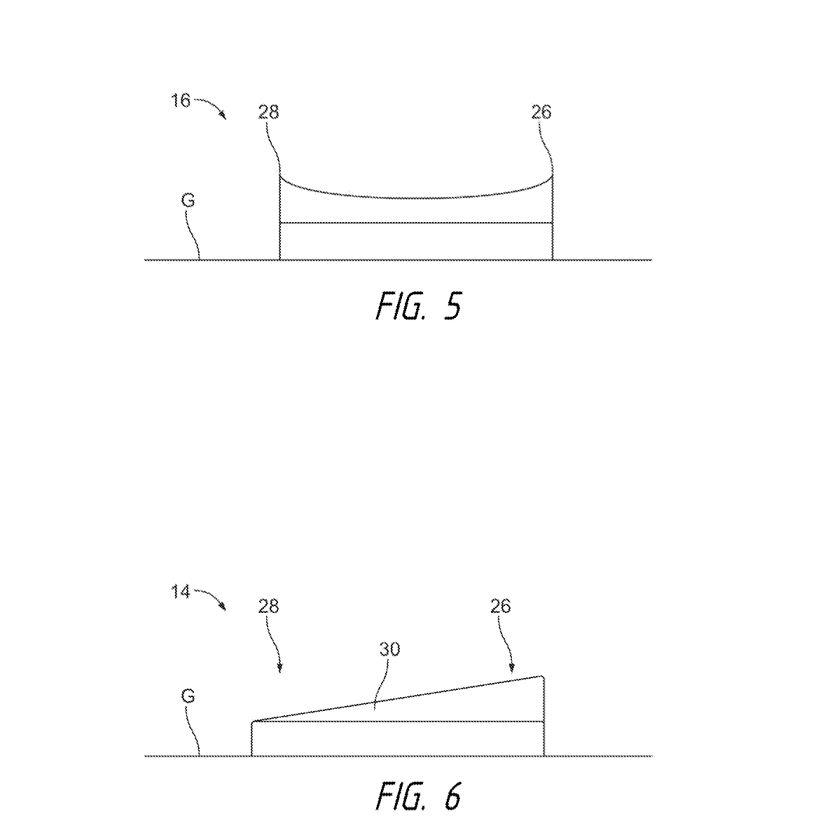

Additionally, as shown in Figure 6, the midfoot section 14 has a midfoot varus 30 of approximately 1-5 degrees. However, variations of the design can include a midfoot varus having a larger range, such as 1-9 degrees. In one example, the varus 30 results from the medial portion 26 of insole being thicker than the lateral portion 28 of the insole, as shown in FIGS. 7A-7F. The midfoot varus 30 of the insole of the midfoot section 14 encourages a transfer of weight from the medial side 26 of the foot to the lateral side 28 of the foot.

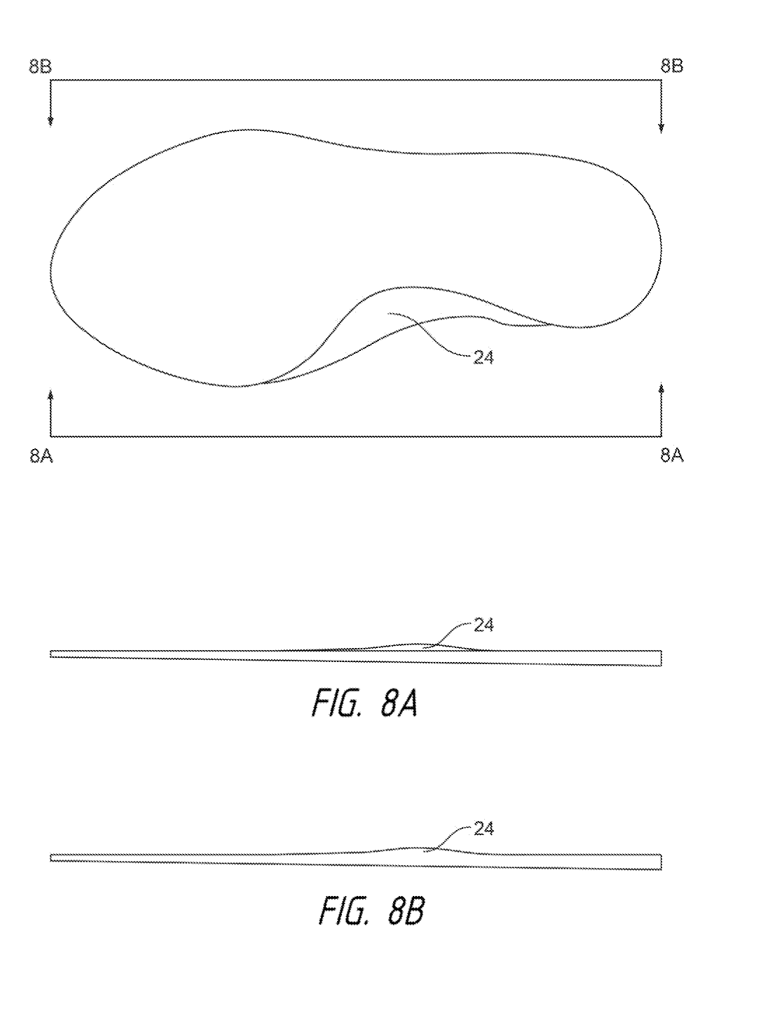

The midfoot section insole 15 may also include an arched portion 24, as shown in FIGS. 8A and 8B. The arched portion defines a raised portion of the insole. The anterior aspect of the arched portion extends superiorly and rearwardly. For example, some variations can extend until reaching a maximum height of 5-30 mm. The arched portion eventually turns inferiorly, continuing to slant in a rearward direction. The arched portion 24 extends along with width of the insole 15 (as shown in the cross-sectional views of FIGS. 8A and 8B) and thus provides support for the medial longitudinal arch MA, the lateral longitudinal arch LA and the transverse arch TA and thus helps to prevent overpronation and plantar fasciitis while providing support for the foot and body.

Further, the exterior of arched portion 24 may be integral with or independent from the insole 80. Likewise, the exterior of arched. portion 24 may be made of the same or different material as that of the insole 80. For example, the exterior material of arched portion 24 may be more rigid than that of the insole or remainder of the shoe and include without limitation materials such as hardened leather.

The insole 80 may be made of one or more materials, and may be permanently or detachably affixed to the shoe. The insole 80 may be a partial insole or extend along the entire length of the shoe. Optionally, the insole 80 of the shoe 10 may be cushioned. The insole 80 can be made from any suitable material which can cushion and absorb the shock experienced by the foot and body during walking. In certain variations, the insole can be made from a single material. Alternatively, the insole can be comprised of multiple pieces, a contiguous piece of material or assembled from multiple pieces. In additional variations, the insole can comprise a permanent part of the shoe and can be built into the shoe. Suitable insole materials can include any commercially available materials.

The outsole 70 of the shoe 10 may be slip resistant and be made of suitable materials such as any polymer or other material used for shoes and/or apparel.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

We claim:

1. A shoe wearable on a foot, the shoe comprising:
a forefoot section;
a midfoot section having a varus causing the midfoot section to slope from a medial side of the midfoot section to a lateral side of the midfoot section; and
a heel section.

2. The shoe of claim 1, wherein the heel section has a varus of 0 degrees.

3. The shoe of claim 1, wherein the midfoot section has a varus of 1 to 5 degrees.

4. The shoe of claim 1 further comprising a slip resistant outsole

5. The shoe of claim 1 further comprising a cushioned insole.

6. The shoe of claim 1 wherein the shoe may include elastic material to accommodate swelling of the foot.

7. The shoe of claim 1, wherein the shoe comprises a partial d'Orsay shape.

8. The shoe of claim 1, wherein the midfoot section further comprises an arched portion, the arched portion configured to provide support for the medial longitudinal, lateral longitudinal, and transverse arches.

9. The shoe of claim 1, wherein the varus causes weight distribution from a medial side of the foot to a lateral side of the foot.

10. A shoe wearable on a foot, the shoe comprising:
a forefoot section;
a midfoot section having a varus causing the midfoot section to slope from a medial side of the midfoot section to a lateral side of the midfoot section, where the varus causes weight distribution from a medial side of the foot to a lateral side of the foot; and
a heel section, wherein a vertical axis of the heel section is perpendicular to a ground plane.

11. A shoe wearable on a foot, the shoe comprising:
a forefoot section;
a midfoot section having a varus causing the midfoot section to slope from a medial side of the midfoot section to a lateral side of the midfoot section, where the varus causes weight distribution from a medial side of the foot to a lateral side of the foot,
the midfoot section further comprising an arched portion, the arched portion configured to provide support for the medial longitudinal, lateral longitudinal, and transverse arches;
a heel section; and
wherein the shoe comprises a partial d'Orsay shape.

12. The shoe of claim 11, wherein the heel section has a varus of 0 degrees.

13. The shoe of claim 11, wherein the midfoot section has a varus of 1 to 5 degrees.

14. The shoe of claim 11, further comprising a slip resistant sole

15. The shoe of claim 11, further comprising a cushioned insole.

16. The shoe of claim 11, wherein the shoe may include elastic material to accommodate swelling of the foot.
